(19) United States
(12) Patent Application Publication
(10) Pub. No.: US 2010/0042030 A1
(43) Pub. Date: Feb. 18, 2010

(54) Dual-bracket cervical brace

(76) Inventor: Howard R. Barnes, Bloomsbury, NJ (US)

Correspondence Address: THOMAS J. GERMINARIO, ESQ., 154 ROUTE 206, CHESTER, NJ, 07930, US

(21) Appl. No.: 12/461,523

(22) Filed: Aug. 14, 2009

Related U.S. Application Data
(60) Provisional application No. 61/189,271, filed on Aug. 15, 2008.

(51) Int. Cl. A61F 5/00

(52) U.S. Cl. 602/18

(57) ABSTRACT

A dual-bracket cervical brace provides frontal support between the chin and sternum with a hinged chin support. This brace allows lateral movements of the wearer's head and neck as well as vertical movements of the head and jaw.

### CROSS REFERENCE TO RELATED APPLICATIONS

This application claims the benefit of the filing date of U.S. Provisional Patent Application No. 61/189,271, which was filed on Aug. 15, 2008.

### BACKGROUND OF THE INVENTION

The present invention relates to cervical and occipital braces for use by individuals who, due to disease or trauma, have impaired muscular strength in the neck and have difficulty supporting their head in an upright position.

Several diseases, including amyotrophic lateral sclerosis (ALS), result in progressive atrophy and/or weakening of the neck muscles, which makes upright support for the head in a standing or sitting posture difficult and fatiguing. Similar muscular atrophy and/or weakening of the neck muscles can result from trauma. For individuals suffering from these conditions, there is a need for a cervical brace that will reinforce frontal support for the head and chin without unduly impairing lateral and vertical mobility of the head, neck and jaw.

The prior art cervical and occipital braces do not fulfill this need. Full cervical collars that encircle the neck, such as the one disclosed by Kohnke, U.S. Pat. No. 5,797,863, are unduly restrictive with respect to the wearer's lateral and vertical head and neck movements. Other neck braces, such as the one taught by Bugarin, U.S. Pat. No. 6,409,694, provide posterior occipital support, but lack the requisite frontal support between the chin and sternum. While the head brace disclosed by MacNeil, U.S. Pat. No. 3,779,549, does provide frontal support between the chin and sternum, it is designed to restrain downward vertical movement of the wearer's head. Similarly, the braces taught by the patents of Kaiser et al. and Hayner, U.S. Pat. Nos. 2,284,058 and 3,455,300, while providing frontal neck support between the chin and sternum, lack a mechanism for allowing downward movements of the head and jaw.

The present invention, on the other hand, is a dual-bracket cervical brace providing frontal support between the chin and sternum with a hinged chin support. This brace allows lateral movements of the wearer's head and neck as well as vertical movements of the head and jaw.

### SUMMARY OF THE INVENTION

The present invention comprises two cervical brackets, which are integrally joined on their distal ends by a lateral sternum strut to form a U-shaped dual-bracket truss. At their proximal ends, the cervical brackets are hingeably connected by two hinge structures to either end of a horizontal chin support, such that the cervical brackets, sternum strut and chin support together form a continuous loop. The cervical brackets, sternum strut and chin support comprise curvilinear, rigid metal or plastic rods, which preferably are covered with a plastic, fabric and/or other pliant padding material for the comfort of the wearer. The hinge structures comprise springs or elastically bendable, semi-rigid rod segments of plastic or steel rods.

In order to retain the brace in position between the chin and sternum, a neck band is slideably attached to the two cervical brackets. The neck band has an adjustable closure means, such as cooperating Velcro strips, so that it may be snuggly placed around the wearer's neck.

The present invention therefore provides frontal support for the wearer's head while permitting lateral rotational movement of the head and neck and vertical up-down movements of the head and jaw.

### BRIEF DESCRIPTION OF THE DRAWING

FIG. 1 is a perspective view of a dual-bracket cervical brace in accordance with the preferred embodiment of the present invention.

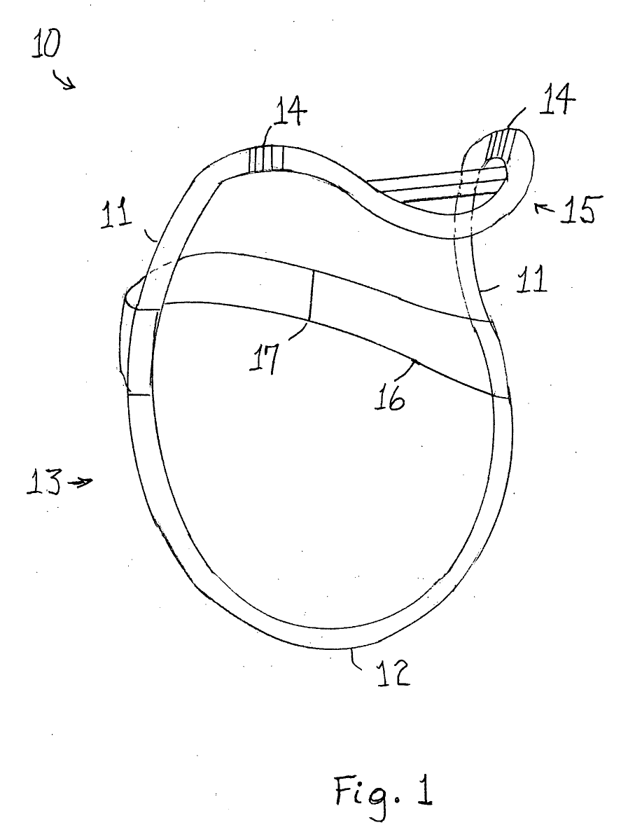

### DETAILED DESCRIPTION OF THE PREFERRED EMBODIMENT

The present invention 10 comprises two cervical brackets 11, which are integrally joined on their distal ends by a lateral sternum strut 12 to form a U-shaped dual-bracket truss 13. At their proximal ends, the cervical brackets 11 are hingeably connected by two hinge structures 14 to either end of a horizontal chin support 15, such that the cervical brackets 11, sternum strut 12 and chin support 15 together form a continuous loop. The cervical brackets 11, sternum strut 12 and chin support 15 comprise curvilinear, rigid metal or plastic rods, which preferably are covered with a plastic, fabric and/or other pliant padding material for the comfort of the wearer. The hinge structures 14 comprise springs or elastically bendable, semi-rigid rod segments of plastic or steel rods.

In order to retain the brace in position between the chin and sternum, a neck band 16 is slideably attached to the two cervical brackets 11. The neck band 16 has an adjustable closure means 17, such as cooperating Velcro strips, so that it may be snuggly placed around the wearer's neck.

Although a preferred embodiment of the invention has been disclosed for illustrative purposes, those skilled in the art will appreciate that many additions, modifications and substitutions are possible, without departing from the scope and spirit of the present invention as defined by the accompanying claims.

What is claimed is:

1. A cervical brace for providing frontal support for a wearer's neck, which cervical neck brace is insertable between the wearer's chin and sternum, said cervical brace comprising:
(a) a U-shaped dual-bracket truss, comprising two cervical brackets, which are integrally joined at their distal ends by a lateral sternum strut, wherein the cervical brackets and sternum strut comprise a continuous curvilinear, rigid metal or plastic rod;
(b) a horizontal chin support, comprising a curvilinear, rigid metal or plastic rod, the ends of which are hingeably connected by two hinge structures to the proximal ends of the two cervical brackets, such that the cervical brackets, sternum strut and chin support together form a continuous loop; and
(c) a neck band, the ends of which are slideably attached to the two cervical brackets, such that the neck band can be placed around the wearer's neck in order to retain the cervical brace in position between the chin and the sternum.

2. The cervical brace according to claim 1, wherein the neck band has an adjustable closure means, such that the neck band can be snuggly placed around the wearer's neck in order to retain the cervical brace in position between the chin and sternum.

3. The cervical brace according to claim 2, wherein the chin support is covered with a pliant padding material.

4. The cervical brace according to claim 3, wherein the dual-bracket truss is covered with a pliant padding material.

5. The cervical brace according to any one of claims 1-4, wherein the hinge structures comprise springs.

6. The cervical brace according to any one of claims 1-4, wherein the hinge structures comprise elastically bendable, semi-rigid rod segments of plastic or steel rods.
